(19) United States
(12) Patent Application Publication
(10) Pub. No.: US 2003/0039681 A1
(43) Pub. Date: Feb. 27, 2003

(54) Rapid growth dairy heifers having reduced mammary fat content

(76) Inventors: Elliot Block, Yardley, PA (US); William K. Sanchez, Tigard, OR (US); Kenneth R. Cummings, Skillman, NJ (US)

Correspondence Address: WATOV & KIPNES, P.C., P.O. Box 247, Princeton Junction, NJ, 08550, US

(21) Appl. No.: 09/928,624

(22) Filed: Aug. 13, 2001

(51) Int. Cl. A61K 31/202; A23K 1/165

(52) U.S. Cl. 424/442; 514/560

(57) ABSTRACT

The present invention provides a feedstock for intensive feeding of replacement dairy heifers to promote rapid growth, and facilitate early breeding and first calving at an age of about 20-24 months. The calved heifers subsequently yield increased first lactation milk production and lifetime milk. An essential ingredient of the invention feedstock is conjugated linoleic acid derivative having rumen-bypass properties. The quantity of conjugated linoleic acid derivative is effective for prevention of mammary gland tissue damage by minimizing mammary fat content. A preferred feedstock includes a content of slow-release degradable nitrogen source for efficient rumen fermentation, and a content of rumen-bypass protein.

### BACKGROUND OF THE INVENTION

This invention generally relates to nutrient-supplemented feedstocks for dairy replacement heifers. More specifically this invention relates to the provision of a feedstock which is adapted for the rapid growth of prepubertal heifers having increased size and weight, and having reduced mammary fat content at first calving.

Growing dairy replacement heifers to 80-85 percent of their mature size at first calving is important to the production economics of a dairy herd. By increasing growth rate and lowering age at first calving, dairy producers significantly influence the largest variable cost of a dairy enterprise. The challenge is to achieve rapid-growth heifers without adverse effect on mammary development and first lactation yield. A main objective is improved turnover rate among first lactation heifers, and the ability of the heifers to freshen and lactate without disease or weight loss.

To optimize profitability of a dairy enterprise, replacement heifers need to calve at 22-24 months of age, while having a body weight greater than 550 kilograms. Rapid growth with intensive feeding is necessary to meet these criteria.

However, it is well-known that rapid growth in young heifers causes excessive fat deposition in the mammary gland. This results in permanent damage to mammary tissue. In an impaired mammary gland, the parenchyma and the epithelial cell number and size are reduced, and there is a subsequent decrease in milk production. It has been shown that there is 7% less milk for every 100 grams daily gain over the optimal growth rate in young heifers [Rep. 693 Natl. Inst. Anim. Sci.; Foulum, Denmark (1991)].

The conflict between desired early breeding and mammary tissue damage caused by intensive feeding has initiated many investigations. Modified feed regimens have been proposed, such as alternating low and high levels of nutrition stages to obtain compensatory growth of mammary tissue without fat deposition. These modified feed regimens have not accomplished the desired results. The usual practice is feeding young heifers a restricted diet, which delays sexual maturity in the animals. The average age at first calving for Holsteins is 28 months in the United States. This is four months longer than the ideal age of 24 months.

During the period commencing with the onset of puberty through puberty, (age 3-9 months), heifers undergo allometric development, and the mammary gland grows more rapidly than the body mass. This growth in the mammary gland is characterized by rapid expansion of the fat pad and branching of ducts. The type of nutrition during this period critically affects the development of the mammary gland.

Of special interest with respect to the present invention are reported studies in connection with the observed physiological effects of conjugated linoleic acid (CLA) in many health-related areas of human and laboratory animal models.

Conjugated linoleic acid (CLA) is a collective term for a mixture of positional and geometric isomers of linoleic acid. The main constituents are cis-9, trans-11 and trans-10, cis-12 linoleic acids, with a lesser content of cis-8, trans-10 linoleic acid. CLA is produced in ruminant animals during biohydrogenation of C-18 polyunsaturated fatty acids in the rumen. CLA also is produced in animal tissues by delta-9-desaturase activity.

Studies have established that CLA can induce a reduction of body fat accumulation. A CLA-supplement fed to growing pigs decreases lipogenesis in adipose tissue and results in lower carcass lipid content [J. Dairy Sci., 83 (Suppl. 1) 179 (2000)]. CLA-supplementation to lactating dairy cattle by abomasal infusion decreases milk fat content [J. Dairy Sci., 83 (Suppl. 1), 163 (2000)].

Another study reports that CLA inhibits proliferation of preadipocytes, and this finding suggests that CLA inhibits stomal vascular preadipocyte hyperplasia [J. Nutri., 129, 92 (1999)].

There is continuing interest in the development of new and improved additive supplemented diets for enhancing the health and productivity of ruminants and other domesticated animals.

Accordingly, it is an object of this invention to provide a feedstock adapted for the rapid growth of prepubertal dairy heifers having reduced mammary fat content.

It is another object of this invention to provide a feedstock and a means for rapid volumetric growth of dairy heifers for breeding and calving within a 20-22 month period.

It is a further object of this invention to provide a feedstock and diet regimen for calving younger and larger dairy heifers, and achieving an increase in first lactation milk production and lifetime milk.

Other objects and advantages of the present invention shall become apparent from the accompanying description and example.

### DESCRIPTION OF THE INVENTION

One or more objects of the present invention are accomplished by the provision of a feedstock for intensive feeding of dairy heifers, which feedstock comprises (1) nutritionally balanced ration adapted for rapid growth of replacement dairy heifers; and (2) conjugated linoleic acid derivative having rumen-bypass properties, and provided in a quantity which is systemically effective for preventing mammary gland tissue damage by minimizing mammary fat content during growth.

The nutritionally balanced ration component of an invention feedstock is custom formulated for intensive feeding and rapid growth of replacement dairy heifers. A ration component is isocaloric and isonitrogenous, and preferably includes a content of slow-release degradable nitrogen source for efficient rumen fermentation, and a content of rumen-bypass protein.

A typical ration can include about 14-20 percent of crude protein, about 25-35 percent of rumen-bypass protein, and about 1-12 percent of slow-release degradable nitrogen source, based on dry matter. The net carbohydrate and protein system of the ration component of the feedstock is maintained in optimal balance.

The following basal ration illustrates the nutrient constituents suitable for intensive feeding of Holstein heifer calves having an average body weight of 300 pounds, and with a target weight gain of about 800-1000 grams per day and a target DMI of about 4-5 kilograms per day:

| Nutrients | Weight % |
|---|---|
| Soybean meal (49% CP) | 3.25 |
| Oats | 9.30 |
| Molasses | 2.30 |
| Min/vit | 1.40 |
| Grass hay | 16.00 |
| Bypass protein | 3.00 |
| Slow-release NPN | 2.25 |
| Corn silage | 16.00 |
| Cracked corn | 25.00 |
| Hayage | 19.00 |

An essential nutritive ingredient of an invention feedstock is conjugated linoleic acid derivative (CLA) having rumen-bypass properties, and is provided in a quantity which is effective for preventing mammary gland tissue damage during heifer growth. The conjugated linoleic acid derivative effectively minimizing mammary fat content, thereby preventing mammary gland damage which otherwise would occur in the absence of the conjugated linoleic acid derivative under intensive feeding conditions.

With respect to the inhibition of fat synthesis and deposition in the mammary gland tissue of intensively fed heifers, trans-10, cis-12 linoleic acid and cis-8, trans-10 linoleic acid derivatives are effective in reducing the activities of key lipogenic enzymes, and thereby decreasing rates of lipogenesis. The cis-9, trans-11 linoleic acid isomer has little or no effect on the expression of fatty acid synthetase in the heifers.

An essential feature of the conjugated linoleic acid derivative is rumen-bypass capability. The conjugated linoleic acid derivative can be in the form of CLA alkaline earth metal salt, such as calcium and/or magnesium salt. The derivative also can be in the form of CLA amide, in which the amide nitrogen is substituted with hydrogen and/or aliphatic radicals. Fatty acid salts and amides having rumen-bypass properties are described in publications such as U.S. Pat. Nos. 4,642,317; 4,826,694; 5,391,788; 5,425,693; 5,456,927; 5,496,572; 5,547,686; 5,670,191; 5,874,102; and the like, incorporated by reference.

Optionally, the CLA can be rumen-protected when in the form of polymer-encapsulated matrix. Typically, the polymer is a continuous film coating that functions as an impermeable barrier under rumen conditions. Suitable polymers include gum arabic, polyvinylpyrrolidone, polyacrylamide, polyvinyl acetate, cellulose acetate, zein, shellac, and the like.

The conjugated linoleic acid derivative normally is incorporated in an invention feedstock in a quantity between about 1-12 grams/kg dietary DM, depending on the effectiveness of rumen protection.

An optional biologically active ingredient can be included in an invention feedstock in an effective quantity between about 0.05-20 weight percent, based on the dry matter weight of the feedstock. It can be selected from a broad variety of nutrients and medicaments, either as a single component or a mixture of components, which are illustrated by the following listing of active ingredients:

1. acid-base buffers which typically are selected from carbonate and phosphate salts, and which serve to moderate and control the dietary anion-cation difference (DCAD) of an invention feedstock, and to reduce the rate and extent of biohydrogenation of free fatty acids in the rumen.

2. sugars and complex carbohydrates which include both water-soluble and water-insoluble monosaccharides, disaccharides and polysaccharides.

Cane molasses is a byproduct from the extraction of sucrose from sugar cane. It is commercially available at standard 79.5° Brix concentration, which has a water content of about 21 weight percent, and a sugar content of 50 weight percent. Sugar beet byproducts also are available as low cost carbohydrate sources.

Whey is a byproduct of the dairy industry. The whey is a dilute solution of lactalbumin, lactose, fats, and the soluble inorganics from milk. Dried whey solids typically have the following composition:

| | |
|---|---|
| Protein | 12.0% |
| Fat | 0.7% |
| Lactose | 60.0% |
| Phosphorus | 0.79% |
| Calcium | 0.874% |
| Ash | 9.7% |

Another source of carbohydrate is derived from the pulp and paper industry which produces large quantities of byproduct lignin sulfonates from wood during the sulfite pulping process. The carbohydrate byproduct is a constituent of the spent sulfite liquor.

3. aminoacid ingredients either singly or in combination which include arginine, histidine, isoleucine, leucine, lysine, methionine, phenylalanine, threonine, tryptophan, valine, tyrosine ethyl HCl, alanine, aspartic acid, sodium glutamate, glycine, proline, serine, cysteine ethyl HCl, and the like, and analogs and salts thereof.

4. vitamin ingredients either singly or in combination which include thiamine HCl, riboflavin, pyridoxine HCl, niacin, niacinamide, inositol, choline chloride, calcium pantothenate, biotin, folic acid, ascorbic acid, vitamin B₁₂, p-aminobenzoic acid, vitamin A acetate, vitamin K, vitamin D, vitamin E, and the like.

Trace element ingredients include compounds of cobalt, copper, manganese, iron, zinc, tin, nickel, chromium, molybdenum, iodine, silicon, vanadium and selenium.

5. protein ingredients as obtained from sources such as dried blood or meat meal, dried and sterilized animal and poultry manure, fish meal, liquid or powdered egg, fish solubles, cell cream, soybean meal, cottonseed meal, canola meal, and the like.

Protein ingredients include non-protein nitrogen compounds such as urea, biuret, ammonium phosphate, and the like.

6. antioxidants as illustrated by butylated hydroxyanisole, butylated hydroxytoluene, tocopherol, tertiary-butylhydroquinone, propyl gallate, and ethoxyquin; and suitable preservatives include sodium sorbate, potassium sorbate, sodium benzoate, propionic acid, α-hydroxybutyric acid, and the like.

7. suspension stabilizing agents which preferably are selected from nonionic surfactants, hydrocolloids and cellulose ethers. These types of chemical agents are illustrated by polyethylene oxide condensates of phenols, C8-C22 alcohols and amines; ethylene oxide reaction products with fatty acid partial esters of hexitans; alkylarylpolyoxyethylene glycol phosphate esters; gum arabic; carob bean gum; tragacanth gum; ammonium, sodium, potassium and calcium alginates; glycol alginates; xanthan gum; potato agar; alkylcellulose; hydroxyalkylcellulose; carboxyalkylcellulose; and the like.

In another embodiment this invention provides a method for intensive feeding of dairy heifers which comprises providing replacement dairy heifers with a feedstock comprising (1) nutritionally balanced ration adapted for rapid growth of dairy heifers; and (2) conjugated linoleic acid derivative having rumen-bypass properties, and provided in a quantity which is systemically effective for prevention of mammary gland tissue damage by minimizing mammary fat content during growth.

The feeding regimen typically is at a rate between about 3-8 Kg/heifer/day as sufficient to sustain rapid growth.

The rate of feeding the heifers preferably is controlled to provide between about 3-30 grams/heifer/day of active conjugated linoleic acid derivative as sufficient to prevent mammary gland tissue damage. The amount of rumen-bypass conjugated linoleic acid derivative in the intestine preferably is between 2-20 grams/heifer/day.

Optimal results are achieved when the intensive feeding period is inclusive of the prepubertal 3-9 month growth phase of the dairy heifers.

The health and growth rate of dairy heifers can be promoted by the inclusion of protein and nonprotein nitrogen additives. FERMENTEN™ (Biovance Technologies; Omaha, Nebr.) is a blend of crude protein, by-pass protein and nonprotein nitrogen composition (U.S. Pat. No. 5,709,894). OPTIGEN™ 1200 (Agway; Dewitt, N.Y.) is a slow-release encapsulated urea formulation (U.S. Pat. No. 6,231,895).

To facilitate the accomplishment of target objectives, the present invention provides a further embodiment which corresponds to a nitrogen-rich nutrient formulation composed of ingredients comprising (1) byproduct-solids recovered from fermentation liquor; and (2) between about 0.5-20 weight percent of an additive ingredient comprising conjugated linoleic acid derivative having rumen-bypass properties.

Illustrative of byproduct-solids are glutamic acid fermentation solubles and/or corn fermentation solubles.

It is particularly preferred that the byproduct-solids are recovered from a heat-treated fermentation liquor. A nitrogen-rich nutrient formulation as defined herein can be derived by blending a customized admixture of FERMENTEN™ and conjugated linoleic acid derivative having rumen-bypass properties. A buffer ingredient can be included as appropriate.

Practice of the present invention embodiments provides manifold advantages.

Rapid growth of dairy heifers in accordance with the present invention facilitates early breeding and first calving at an age of about 20-24 months. As a further advantage, the calved dairy heifers subsequently yield increased first lactation milk production and lifetime milk. The desirable advantages of the present invention embodiments mainly derive from the prevention of mammary gland tissue damage by minimizing mammary fat content during the rapid growth phase of the dairy heifers.

The following Example is further illustrative of the present invention. The components and specific ingredients are presented as being typical, and various modifications can be derived in view of the foregoing disclosure within the scope of the invention.

### EXAMPLE

The following Example illustrates the advantages obtained by the rapid growth of replacement heifers in accordance with the present invention.

Ninety Holstein heifers are randomly assigned to receive one of three dietary treatments at 6 months of age and 195 kg of body weight. The three treatments are: 1) Formulation to achieve an average daily body weight gain (ADG) of 1.0 kg/day with the addition of 68 g/animal/day of Conjugated Linoleic Acid Amide (CLA-Amide); 2) Same formulation as treatment #1 with Megalac™ in place of CLA-Amide; 3) Formulation to achieve an ADG of 700 g/day. Diets are fed until heifers achieve a body weight of 360 kg at which time all heifers are hormonally synchronized with the OvSynch program and bred. From breeding to calving all heifers are fed identical diets formulated to achieve an ADG of 700 g/day.

The three diets are summarized in TABLES I and II, with nutrient requirements obtained from NRC, 2001.

The free CLA from which the amide form is produced contains 60% CLA of which 50% is the cis-9, trans-11 isomer and 50% is the trans-10, cis-12 isomer. The free CLA is mixed with ammonia under pressure to produce the corresponding amides. On this basis 68 g of this CLA-Amide delivers 20.4 g/d of each isomer to the rumen of heifers of which 80 to 85% is considered ruminally inert and bypasses to the small intestine. Heifers fed treatment 1 receive between 16.4 and 17 g/d of each CLA isomer in the intestine.

Immediately after calving 10 randomly selected heifers from each group are sacrificed. The mammary glands are removed, emptied of milk, weighed, homogenized and analyzed for total DNA content. The remaining 20 heifers in each group are fed identical rations formulated for normal growth rates and high milk production (NRC, 2001) for their first 100 days in lactation. Daily milk production, weekly milk composition for fat and protein and monthly body weights are measured and recorded.

All data except mammary gland parameters are analyzed by the repeated measures analysis of the Statistical Analysis System (SAS). Mammary data are analyzed as a randomized design by the General Linear Models (GLM) procedure using SAS.

### RESULTS

Heifers consuming Diets 1 and 2 reach their target body weights for breeding by 11.4±0.04 months of age with no age difference (P>0.4) between the two groups. Heifers consuming diet 3 reach their target body weight by 15±0.3 months of age which is at an older age (P<0.01) than heifers fed diets 1 and 2.

Average age at calving for heifers fed Diets 1 and 2 is 21.2±0.5 months with no difference (P>0.4) between the two groups. Average age at calving for heifers fed Diet 3 is 24.5±0.5 months which is older (P<0.01) than heifers fed Diets 1 and 2.

Total emptied mammary gland weight in sacrificed heifers is the same (P>0.2) for heifers fed Diets 1 and 3. Heifers fed Diet 2 have heavier (P<0.05) mammary glands by 15% than heifers fed Diets 1 and 2. Homogenized mammary gland samples from heifers fed Diet 2 have 22% more (P<0.01) ether extractable, fat and 29% less (P<0.01) total DNA than heifers fed Diets 1 and 2, with no differences (P>0.2) between heifers fed Diets 1 and 2. These results show that when CLA is delivered to fast growing heifers, equal mammary growth can be achieved as when heifers are grown slower than currently recommended growth rates. Without the CLA the faster growth rates result in heavier glands that contain more fat and less secretory tissue than either slower growth rates or faster growth rates with CLA.

Average daily milk production for heifers fed Diets 1, 2 and 3 is 41.1±0.4, 34.2±0.5 and 40.6±0.6 kg/day/heifer. Heifers fed Diet 2 produce less (P<0.01) milk than those fed Diets 1 and 3 with no difference (P>0.3) in production between heifers fed Diets 1 and 3. Milk fat and protein composition did not differ between treatment groups (P>0.3). Heifers fed Diet 2 product 665 kg less milk (P<0.01) in their first 100 days in milk than heifers fed Diets 1 and 3. Projected milk yield for heifers fed Diets 1 and 3 is 9,240 kg in 305 days while heifers fed Diet 2 calculate at 7,700 kg or 17% less milk. Comparing heifers fed Diet 1 with those fed Diet 3, heifers fed Diet 1 produced the same amount of milk 90 days sooner than those fed Diet 3.

TABLE I
DIET 1 AND DIET 2* (rapid growth)

| | kg/d | Kg/d | | |
|---|---|---|---|---|
| Ingredient | AF¹ | DM² | % AF | % DM |
| Fermenten ™ | 0.556 | 0.50 | 4.19 | 9.77 |
| Prolak ™ | 0.045 | 0.04 | 0.34 | 0.82 |
| CLA-AMIDE* | 0.070 | 0.068 | 0.53 | 1.33 |
| Ground corn | 2.026 | 1.78 | 15.27 | 34.79 |
| Grass silage | 10.441 | 2.61 | 78.71 | 50.94 |
| Min/vit. mix | 0.110 | 0.10 | 0.83 | 2.03 |

*Same as Diet 1, with Megalac ™ substituted for CLA-AMIDE ingredient.
¹As fed.
²Dry matter.

DIET 1 AND DIET 2

| Feedstock Values | Unit | DM |
|---|---|---|
| Dry matter | % | 100.00 |
| Crude protein | % | 17.07 |
| Undegradable protein | % CP | 29.26 |
| Degradable protein | % CP | 70.74 |
| Soluble protein | % CP | 46.46 |
| Metabolizable energy | mCal/kg | 2.74 |
| Net energy maintenance | mCal/kg | 1.82 |
| Net energy gain | mCal/kg | 1.19 |
| Neutral detergent fiber | % | 34.25 |
| Total fat | % | 4.66 |
| Calcium | % | 0.42 |
| Phosphorus | % | 0.42 |
| Magnesium | % | 0.14 |
| DCAD (dietary cation-anion difference) | meg/100 g | 18.36 |

TABLE II
DIET 3 (slow growth)

| | kg/d | kg/d | | |
|---|---|---|---|---|
| Ingredient | AF | DM | % AF | % DM |
| Fermenten ™ | 0.506 | 0.46 | 4.41 | 9.93 |
| Prolak ™ | 0.052 | 0.05 | 0.46 | 1.07 |
| Ground corn | 1.202 | 1.06 | 10.49 | 23.09 |
| Soybean meal (49% CP) | 0.013 | 0.01 | 0.11 | 0.26 |
| Corn silage | 3.936 | 1.38 | 34.34 | 30.07 |
| Grass silage | 5.486 | 1.37 | 47.86 | 29.93 |
| Min/vit. mix | 0.264 | 0.25 | 2.33 | 5.65 |

DIET 3

| Feedstock Values | Unit | DM |
|---|---|---|
| Dry matter | % | 100.00 |
| Crude protein | % | 15.75 |
| Undegradable protein | % CP | 28.35 |
| Degradable protein | % CP | 71.65 |
| Soluble protein | % CP | 48.88 |
| Metabolizable energy | mCal/kg | 2.49 |
| Net energy maintenance | mCal/kg | 1.59 |
| Net energy gain | mCal/kg | 0.99 |
| Neutral detergent fiber | % | 35.11 |
| Total fat | % | 3.18 |
| Calcium | % | 1.37 |
| Phosphorus | % | 0.62 |
| Magnesium | % | 0.84 |
| DCAD | meg/100 g | 15.00 |

What is claimed is:

1. A feedstock for intensive feeding of dairy heifers which comprises (1) nutritionally balanced ration adapted for rapid growth of replacement dairy heifers; and (2) conjugated linoleic acid derivative having rumen-bypass properties, and provided in a quantity which is systemically effective for preventing mammary gland tissue damage by minimizing mammary fat content during growth.

2. A feedstock in accordance with claim 1 wherein the ration has a content of slow-release degradable nitrogen source for efficient rumen fermentation, and/or a content of rumen-bypass protein.

3. A feedstock in accordance with claim 1 wherein the quantity of conjugated linoleic acid derivative is in the range between about 1-12 grams/kg dietary DM.

4. A feedstock in accordance with claim 1 wherein the conjugated linoleic acid is in the form of calcium salt and/or magnesium salt and/or carboxylic acid amide and/or polymer-encapsulated matrix.

5. A feedstock in accordance with claim 1 wherein the systemically effective constituent of the conjugated linoleic acid derivative comprises trans-10, cis-12 structural isomer.

6. A method for intensive feeding of dairy heifers which comprises providing replacement dairy heifers with a feedstock comprising (1) nutritionally balanced ration adapted for rapid growth of dairy heifers; and (2) conjugated linoleic acid derivative having rumen-bypass properties, and provided in a quantity which is systemically effective for prevention of mammary gland tissue damage by minimizing mammary fat content during growth.

7. A method if accordance with claim 6 wherein the feedstock has a content of FERMENTEN™ nitrogen source which is effective for promoting rapid growth of the dairy heifers.

8. A method in accordance with claim 6 wherein the feedstock content of conjugated linoleic acid derivative is between about 1-12 grams/kg dietary DM.

9. A method in accordance with claim 6 wherein the feeding regimen is at a rate between about 3-8 kg/heifer/day as sufficient to sustain rapid growth.

10. A method in accordance with claim 6 wherein the feeding is at a rate which provides between about 3-30 grams/heifer/day of active conjugated linoleic acid derivative as sufficient to prevent mammary gland tissue damage during growth.

11. A method in accordance with claim 6 wherein the intensive feeding period is inclusive of the prepubertal 3-9 month growth phase of the dairy heifers.

12. A method in accordance with claim 6 wherein the rapid growth of the dairy heifers facilitates early breeding and first calving at an age of about 20-24 months, and wherein the dairy heifers subsequently yield increased first lactation milk production and lifetime milk.

13. A nitrogen-rich nutrient formulation which comprises (1) byproduct-solids recovered from fermentation liquor; and (2) between about 0.5-20 weight percent of an additive ingredient comprising conjugated linoleic acid derivative having rumen-bypass properties.

14. A formulation in accordance with claim 13 wherein the byproduct-solids are glutamic acid fermentation solubles and/or corn fermentation solubles.

15. A formulation in accordance with claim 13 wherein the byproduct-solids are recovered from a heat-treated fermentation liquor.

16. A formulation in accordance with claim 13 wherein the byproduct solids are FERMENTEN™ product or a generic equivalent thereof.

17. A formulation in accordance with claim 13 wherein the conjugated linoleic acid is in the form of calcium salt and/or magnesium salt and/or carboxylic acid amide and/or polymer-encapsulated matrix.

18. A formulation in accordance with claim 13 wherein the conjugated linoleic acid derivative comprises tran-10, cis-12 structural isomer.

19. A formulation in accordance with claim 13 which has a content of pH buffer additive.

20. A formulation in accordance with claim 13 which is an additive in an animal feedstock.
